SEQUENCE LISTING (89 sequences)

SEQ ID NO: 1 - protein, 10 aa, Artificial Sequence
  delta-V1-1, having a portion of the amino acid sequence of rat delta-PKC
SFNSYELGSL

SEQ ID NO: 2 - protein, 11 aa, Artificial Sequence
  delta-PKC from rat, amino acids 35 to 45 as found in Genbank Accession No. AAH76505
ALTTDRGKTLV

SEQ ID NO: 3 - protein, 14 aa, Artificial Sequence
  delta-PKC from rat, amino acids 101 to 114 as found in Genbank Accession No. AAH76505
KAEFWLDLQPQAKV

SEQ ID NO: 4 - protein, 58 aa, Artificial Sequence
  Synthetic Peptide
PFRPKVKSPRPYSNFDQEFLNEKARLSYSDKNLIDSMDQSAFAGFSFVNPKFEHLLED

SEQ ID NO: 5 - protein, 10 aa, Artificial Sequence
  Synthetic Peptide
TFNSYELGSL

SEQ ID NO: 6 - protein, 10 aa, Artificial Sequence
  Synthetic Peptide
AFNSYELGSL

SEQ ID NO: 7 - protein, 10 aa, Artificial Sequence
  Synthetic Peptide
SFNSYELGTL

SEQ ID NO: 8 - protein, 10 aa, Artificial Sequence
  Synthetic Peptide
TFNSYELGTL

SEQ ID NO: 9 - protein, 10 aa, Artificial Sequence
  Synthetic Peptide
SYNSYELGSL

SEQ ID NO: 10 - protein, 10 aa, Artificial Sequence
  Synthetic Peptide
SFNSFELGSL

SEQ ID NO: 11 - protein, 9 aa, Artificial Sequence
  Synthetic Peptide
SNSYDLGSL

SEQ ID NO: 12 - protein, 10 aa, Artificial Sequence
  Synthetic Peptide
SFNSYELPSL

SEQ ID NO: 13 - protein, 10 aa, Artificial Sequence
  Synthetic Peptide
SFNSYEIGSV

SEQ ID NO: 14 - protein, 10 aa, Artificial Sequence
  Synthetic Peptide
SFNSYEVGSI

SEQ ID NO: 15 - protein, 10 aa, Artificial Sequence
  Synthetic Peptide
SFNSYELGSV

SEQ ID NO: 16 - protein, 10 aa, Artificial Sequence
  Synthetic Peptide
SFNSYELGSI

SEQ ID NO: 17 - protein, 10 aa, Artificial Sequence
  Synthetic Peptide
SFNSYEIGSL

SEQ ID NO: 18 - protein, 10 aa, Artificial Sequence
  Synthetic Peptide
SFNSYEVGSL

SEQ ID NO: 19 - protein, 10 aa, Artificial Sequence
  Synthetic Peptide
AFNSYELGSL

SEQ ID NO: 20 - protein, 6 aa, Artificial Sequence
  Synthetic Peptide
YELGSL

SEQ ID NO: 21 - protein, 6 aa, Artificial Sequence
  Synthetic Peptide
YDLGSL

SEQ ID NO: 22 - protein, 6 aa, Artificial Sequence
  Synthetic Peptide
FDLGSL

SEQ ID NO: 23 - protein, 6 aa, Artificial Sequence
  Synthetic Peptide
YDIGSL

SEQ ID NO: 24 - protein, 4 aa, Artificial Sequence
  Synthetic Peptide
IGSL

SEQ ID NO: 25 - protein, 6 aa, Artificial Sequence
  Synthetic Peptide
YDVGSL

SEQ ID NO: 26 - protein, 6 aa, Artificial Sequence
  Synthetic Peptide
YDLPSL

SEQ ID NO: 27 - protein, 6 aa, Artificial Sequence
  Synthetic Peptide
YDLGIL

SEQ ID NO: 28 - protein, 6 aa, Artificial Sequence
  Synthetic Peptide
YDLGSI

SEQ ID NO: 29 - protein, 6 aa, Artificial Sequence
  Synthetic Peptide
YDLGSV

SEQ ID NO: 30 - protein, 4 aa, Artificial Sequence
  Synthetic Peptide
LGSL

SEQ ID NO: 31 - protein, 4 aa, Artificial Sequence
  Synthetic Peptide
IGSL

SEQ ID NO: 32 - protein, 4 aa, Artificial Sequence
  Synthetic Peptide
VGSL

SEQ ID NO: 33 - protein, 4 aa, Artificial Sequence
  Synthetic Peptide
LPSL

SEQ ID NO: 34 - protein, 4 aa, Artificial Sequence
  Synthetic Peptide
LGIL

SEQ ID NO: 35 - protein, 4 aa, Artificial Sequence
  Synthetic Peptide
LGSI

SEQ ID NO: 36 - protein, 4 aa, Artificial Sequence
  Synthetic Peptide
LGSV

SEQ ID NO: 37 - protein, 11 aa, Artificial Sequence
  Synthetic Peptide
ALSTDRGKTLV

SEQ ID NO: 38 - protein, 11 aa, Artificial Sequence
  Synthetic Peptide
ALTSDRGKTLV

SEQ ID NO: 39 - protein, 11 aa, Artificial Sequence
  Synthetic Peptide
ALTTDRGKSLV

SEQ ID NO: 40 - protein, 11 aa, Artificial Sequence
  Synthetic Peptide
ALTTDRPKTLV

SEQ ID NO: 41 - protein, 11 aa, Artificial Sequence
  Synthetic Peptide
ALTTDRGRTLV

SEQ ID NO: 42 - protein, 11 aa, Artificial Sequence
  Synthetic Peptide
ALTTDKGKTLV

SEQ ID NO: 43 - protein, 11 aa, Artificial Sequence
  Synthetic Peptide
ALTTDKGKTLV

SEQ ID NO: 44 - protein, 14 aa, Artificial Sequence
  Synthetic Peptide
RAEFWLDLQPQAKV

SEQ ID NO: 45 - protein, 14 aa, Artificial Sequence
  Synthetic Peptide
KADFWLDLQPQAKV

SEQ ID NO: 46 - protein, 14 aa, Artificial Sequence
  Synthetic Peptide
KAEFWLELQPQAKV

SEQ ID NO: 47 - protein, 14 aa, Artificial Sequence
  Synthetic Peptide
KAEFWLDLQPQARV

SEQ ID NO: 48 - protein, 14 aa, Artificial Sequence
  Synthetic Peptide
KAEYWLDLQPQAKV

SEQ ID NO: 49 - protein, 14 aa, Artificial Sequence
  Synthetic Peptide
KAEFWIDLQPQAKV

SEQ ID NO: 50 - protein, 14 aa, Artificial Sequence
  Synthetic Peptide
KAEFWVDLQPQAKV

SEQ ID NO: 51 - protein, 14 aa, Artificial Sequence
  Synthetic Peptide
KAEFWLDIQPQAKV

SEQ ID NO: 52 - protein, 14 aa, Artificial Sequence
  Synthetic Peptide
KAEFWLDVQPQAKV

SEQ ID NO: 53 - protein, 14 aa, Artificial Sequence
  Synthetic Peptide
KAEFWLDLNPQAKV

SEQ ID NO: 54 - protein, 14 aa, Artificial Sequence
  Synthetic Peptide
KAEFWLDLQPNAKV

SEQ ID NO: 55 - protein, 14 aa, Artificial Sequence
  Synthetic Peptide
KAEFWLDLQPQAKI

SEQ ID NO: 56 - protein, 14 aa, Artificial Sequence
  Synthetic Peptide
KAEFWLDLQPQAKL

SEQ ID NO: 57 - protein, 14 aa, Artificial Sequence
  Synthetic Peptide
KAEFWADLQPQAKV

SEQ ID NO: 58 - protein, 14 aa, Artificial Sequence
  Synthetic Peptide
KAEFWLDAQPQAKV

SEQ ID NO: 59 - protein, 14 aa, Artificial Sequence
  Synthetic Peptide
KAEFWLDLQPQAKA

SEQ ID NO: 60 - protein, 7 aa, Artificial Sequence
  Synthetic Peptide
KAEFWLD

SEQ ID NO: 61 - protein, 8 aa, Artificial Sequence
  Synthetic Peptide
DLQPQAKV

SEQ ID NO: 62 - protein, 8 aa, Artificial Sequence
  Synthetic Peptide
EFWLDLQP

SEQ ID NO: 63 - protein, 7 aa, Artificial Sequence
  Synthetic Peptide
LDLQPQA

SEQ ID NO: 64 - protein, 7 aa, Artificial Sequence
  Synthetic Peptide
LQPQAKV

SEQ ID NO: 65 - protein, 7 aa, Artificial Sequence
  Synthetic Peptide
AEFWLDL

SEQ ID NO: 66 - protein, 7 aa, Artificial Sequence
  Synthetic Peptide
WLDLQPQ

SEQ ID NO: 67 - protein, 8 aa, Artificial Sequence
  Synthetic Peptide
SPRPYSNF

SEQ ID NO: 68 - protein, 8 aa, Artificial Sequence
  Synthetic Peptide
RPYSNFDQ

SEQ ID NO: 69 - protein, 8 aa, Artificial Sequence
  Synthetic Peptide
SNFDQEFL

SEQ ID NO: 70 - protein, 8 aa, Artificial Sequence
  Synthetic Peptide
DQEFLNEK

SEQ ID NO: 71 - protein, 8 aa, Artificial Sequence
  Synthetic Peptide
FLNEKARL

SEQ ID NO: 72 - protein, 8 aa, Artificial Sequence
  Synthetic Peptide
LIDSMDQS

SEQ ID NO: 73 - protein, 8 aa, Artificial Sequence
  Synthetic Peptide
SMDQSAFA

SEQ ID NO: 74 - protein, 8 aa, Artificial Sequence
  Synthetic Peptide
DQSAFAGF

SEQ ID NO: 75 - protein, 8 aa, Artificial Sequence
  Synthetic Peptide
FVNPKFEH

SEQ ID NO: 76 - protein, 8 aa, Artificial Sequence
  Synthetic Peptide
KFEHLLED

SEQ ID NO: 77 - protein, 8 aa, Artificial Sequence
  Synthetic Peptide
NEKARLSY

SEQ ID NO: 78 - protein, 8 aa, Artificial Sequence
  Synthetic Peptide
RLSYSDKN

SEQ ID NO: 79 - protein, 8 aa, Artificial Sequence
  Synthetic Peptide
SYSDKNLI

SEQ ID NO: 80 - protein, 8 aa, Artificial Sequence
  Synthetic Peptide
DKNLIDSM

SEQ ID NO: 81 - protein, 8 aa, Artificial Sequence
  Synthetic Peptide
PFRPKVKS

SEQ ID NO: 82 - protein, 8 aa, Artificial Sequence
  Synthetic Peptide
RPKVKSPR

SEQ ID NO: 83 - protein, 8 aa, Artificial Sequence
  Synthetic Peptide
VKSPRPYS

SEQ ID NO: 84 - protein, 17 aa, Artificial Sequence
  Synthetic Peptide
CRQIKIWFQNRRMKWKK

SEQ ID NO: 85 - protein, 11 aa, Artificial Sequence
  Transactivating Regulatory Protein (Tat)-derived transport polypeptide from the Human Immunodeficiency Virus, Type 1
YGRKKRRQRRR

SEQ ID NO: 86 - protein, 673 aa, Rattus norvegicus
MAPFLRISFNSYELGSLQAEDDASQPFCAVKMKEALTTDRGKTLVQKKPTMYPEWKSTFDAHIYEGRVIQIVLMRAAEDPMSEVTVGVSVLAERCKKNNGKAEFWLDLQPQAKVLMCVQYFLEDGDCKQSMRSEEEAMFPTMNRRGAIKQAKIHYIKNHEFIATFFGQPTFCSVCKEFVWGLNKQGYKCRQCNAAIHKKCIDKIIGRCTGTATNSRDTIFQKERFNIDMPHRFKVYNYMSPTFCDHCGSLLWGLVKQGLKCEDCGMNVHHKCREKVANLCGINQKLLAEALNQVTQKASRKPETPETVGIYQGFEKKTAVSGNDIPDNNGTYGKIWEGSNRCRLENFTFQKVLGKGSFGKVLLAELKGKERYFAIKYLKKDVVLIDDDVECTMVEKRVLALAWENPFLTHLICTFQTKDHLFFVMEFLNGGDLMFHIQDKGRFELYRATFYAAEIICGLQFLHGKGIIYRDLKLDNVMLDKDGHIKIADFGMCKENIFGENRASTFCGTPDYIAPEILQGLKYSFSVDWWSFGVLLYEMLIGQSPFHGDDEDELFESIRVDTPHYPRWITKESKDIMEKLFERDPAKRLGVTGNIRLHPFFKTINWNLLEKRKVEPPFKPKVKSPSDYSNFDPEFLNEKPQLSFSDKNLIDSMDQTAFKGFSFVNPKYEQFLE

SEQ ID NO: 87 - protein, 676 aa, Homo sapiens
MAPFLRIAFNSYELGSLQAEDEANQPFCAVKMKEALSTERGKTLVQKKPTMYPEWKSTFDAHIYEGRVIQIVLMRAAEEPVSEVTVGVSVLAERCKKNNGKAEFWLDLQPQAKVLMSVQYFLEDVDCKQSMRSEDEAKFPTMNRRGAIKQAKIHYIKNHEFIATFFGQPTFCSVCKDFVWGLNKQGYKCRQCNAAIHKKCIDKIIGRCTGTAANSRDTIFQKERFNIDMPHRFKVHNYMSPTFCDHCGSLLWGLVKQGLKCEDCGMNVHHKCREKVANLCGINQKLLAEALNQVTQRASRRSDSASSEPVGIYQGFEKKTGVAGEDMQDNSGTYGKIWEGSSKCNINNFIFHKVLGKGSFGKVLLGELKGRGEYFAIKALKKDVVLIDDDVECTMVEKRVLTLAAENPFLTHLICTFQTKDHLFFVMEFLNGGDLMYHIQDKGRFELYRATFYAAEIMCGLQFLHSKGIIYRDLKLDNVLLDRDGHIKIADFGMCKENIFGESRASTFCGTPDYIAPEILQGLKYTFSVDWWSFGVLLYEMLIGQSPFHGDDEDELFESIRVDTPHYPRWITKESKDILEKLFEREPTKRLGVTGNIKIHPFFKTINWTLLEKRRLEPPFRPKVKSPRDYSNFDQEFLNEKARLSYSDKNLIDSMDQSAFAGFSFVNPKFEHLLED

SEQ ID NO: 88 - protein, 8 aa, Artificial Sequence
  delta-PKC peptide, delta-V1-7
MRAAEDPM

SEQ ID NO: 89 - protein, 23 aa, Artificial Sequence
  Synthetic Peptide
YGRKKRRQRRRCCSFNSYELGSL

(19) United States
(12) Patent Application Publication
(10) Pub. No.: US 2009/0062208 A1
(43) Pub. Date: Mar. 5, 2009

(54) Methods for maintaining blood-brain barrier integrity in hypertensive subjects using a delta-PKC inhibitor

(76) Inventors: Daria D. Mochly-Rosen, Melo Park, CA (US); Xin Qi, Mountain View, CA (US); Koichi Inagaki, Otsu (JP)

Correspondence Address: PERKINS COIE LLP, P.O. BOX 1208, SEATTLE, WA, 98111-1208, US

(21) Appl. No.: 12/012,862

(22) Filed: Feb. 5, 2008

Related U.S. Application Data
(60) Provisional application No. 60/899,917, filed on Feb. 6, 2007.

(51) Int. Cl. A61K 38/08; A61K 38/02

(52) U.S. Cl. 514/15; 514/2

(57) ABSTRACT

Methods for maintaining the integrity of the blood-brain barrier are described. Compounds that act to inhibit the action of the delta isozyme of protein kinase C (PKC) to prevent disruption of the blood-brain barrier in hypertensive subjects are described, to, in one embodiment, decrease the likelihood of hypertension-induced stroke or hypertension-induced encephalopathy.

### CROSS-REFERENCE TO RELATED APPLICATIONS

This application claims the benefit of U.S. provisional application Ser. No. 60/899,917, filed Feb. 6, 2007, which is incorporated by reference herein.

### STATEMENT REGARDING GOVERNMENT INTEREST

This work was supported in part by the National Institute of Health Grant number R01 NS 44350. This work was also supported in part by National Institute of Health Grant number R01 HL 076675. Accordingly the United States government has certain rights in this invention.

### TECHNICAL FIELD

The subject matter described herein relates to treatment methods for maintaining the integrity of the blood brain barrier in a hypertensive patient, and thereby reducing cerebral damage in hypertension-induced stroke and encephalopathy. More particularly, the subject matter relates to a method of inhibiting disruption of the blood-brain barrier by administering a delta protein kinase C (δPKC) inhibitor to hypertensive patients.

### BACKGROUND

Hypertension is a public health issue in industrialized nations, affecting approximately 20% of adults. The close associations of hypertension with stroke, atherosclerosis, coronary and cerebrovascular disease, diabetes, and end-stage renal disease make it a major contributor to morbidity and mortality in adult populations. Accumulating evidence suggests that hypertension is responsible for cognitive decline beyond being a leading factor in stroke.

Pathologic changes in the brain and its vasculature that are associated with hypertension include vascular remodeling, impaired cerebral autoregulation, cerebral microbleeds, white matter lesions, unrecognized lacunar infarcts, and Alzheimer-like changes such as amyloid angiopathy and cerebral atrophy. Further pathological features include cerebral infarction, edema, hemorrhage, and encephalopathy due to the breakdown of the blood brain barrier (BBB), a network of tightly-sealed blood vessels in the brain that separates the tissues of the central nervous system from the systemic blood supply.

Numerous transporter and regulatory proteins have been identified in the BBB, including isoforms of the glucose transporter (e.g., GLUT1), protein kinase C (PKC), and caveolin-1. Isoform 1 of the facilitative glucose transporter (GLUT1) is expressed primarily in endothelial (and pericyte) cells. In humans, approximately 75% of the protein is membrane-localized. PKC co-localizes with GLUT1, suggesting an association of PKC with BBB glucose transporter expression. The tight-junction proteins occludin and claudin-5 are expressed within interendothelial clefts of the BBB, as well as glial fibrillary acidic protein (GFAP), monocarboxylic acid transporter and water channel (aquaporin-4) (Cornford, E. M. and Hyman, S. (2005) NeuroRx 2:27-43).

Members of the mammalian PKC superfamily of serine/theronine kinases are known to play roles in a variety of cellular processes. However, a thorough understanding of the mammalian PKC signaling systems has been complicated by the large number of family members. The PKC family includes ten different isozymes, i.e., isozymes α, β, δ, ε, ζ, λ, ι, θ, \acute{η}, and μ, which are comprised of homologous amino acid sequence building blocks (reviewed in, e.g., Mellor, H. and Parker, P. J., Biochem. J., 332:281-292 (1998)). Several PKC isozymes mediate unique cellular functions in response to cellular stresses such as ischemia. Delta PKC (δPKC), in particular, mediates cellular damage following ischemic/reperfusion damage in multiple organs. (La Porta, C. A. et al., Biochem Biophys Res Commun., 191:1124-30 (1993); Bright, R. et al., J. Neurosci., 24:6880-88 (2004)). Inhibition or reduction of δPKC levels during reperfusion leads to marked reduction in cerebral damage. (Bright, R. et al., J. Neurosci. 24:6880-88 (2004); Raval, A. P. et al., J. Cereb. Blood Flow Metab. 25:730-41 (2005)). This protection is due to inhibition of deleterious δPKC activity in multiple cell types including parenchymal and inflammatory cells following stroke (Bright, R. et al., J. Neurosci. 24:6880-88 (2004); Chou, W. H., et al., J. Clin. Invest. 114:49-56 (2004)). In addition, the levels of δPKC increase in endothelial cells 2-3 days following ischemic/reperfusion injury in vivo (Miettinen, S. et al., J. Neurosci. 16:6236-45 (1996)). Whether δPKC specifically mediates acute cerebrovascular responses during reperfusion injury, thereby contributing to stroke damage, is unknown.

The Dahl salt-sensitive (DS) rat is an animal model for study of salt-induced hypertension. DS rats appear to have a genetic functional derangement in the kidney that causes salt retention, although accumulation of sodium in blood or tissues has not been conclusively demonstrated. Switching the DS animals from a low-salt diet (e.g., 1% NaCl) to a high-salt diet (e.g., >8% NaCl) causes the rapid onset of severe hypertension (Werber, A. H. and Fitch-Burke, M. C., Hypertension 12:549-55 (1988)). DS rats fed an 8.7% sodium chloride diet from weaning spontaneously develop hypertension with 50% mortality by 5 weeks. The rats also exhibit behavioral signs of stroke and disruption of the blood-brain barrier. Stroke in DS rats is associated with an inability of the middle cerebral arteries to constrict in response to pressure. The PKC inhibitors chelerythrine and bisindolylmaleimide inhibited constriction, suggesting that middle cerebral artery constriction in response to elevated blood pressure is dependent on functional PKC signaling (Payne, G. W. and Smeda, J. S., J. Hypertens. 20:1355-63 (2002)). Yet, another study demonstrated that increased fluid phase endocytotosis in human brain capillary endothelium, an event thought to be linked to the observed increases in blood-brain barrier permeability in acute hypertension, is likely independent of PKC (Stanimirovic, D. et al., J. Cell Physiol. 169:455-67 (1996)). Therefore, while PKC has been implicated in blood-brain barrier permeability, its role is unclear.

### BRIEF SUMMARY

The following aspects and embodiments thereof described and illustrated below are intended to be exemplary and illustrative, not limiting in scope.

In one aspect, a method is provided for inhibiting disruption of the blood-brain barrier, comprising administering to a patient suffering from hypertension an inhibitor of delta protein kinase C (δPKC). In some embodiments, the inhibitor of δPKC is a peptide. In other embodiments, the peptide is selected from the first variable region of δPKC. In particular embodiments, the peptide is a peptide having between about 5 and 15 contiguous residues from the first variable region of δPKC. In other embodiments, the peptide has at least about 50% sequence identity with a conserved set of between about 5 and 15 contiguous residues from the first variable region of δPKC. In other embodiments the peptide has at least about 80% sequence identity with the δPKC peptide inhibitor SFNSYELGSL (SEQ ID NO:1).

In some embodiments, the peptide is modified to include a carrier molecule. In particular embodiments, the peptide is modified to include an N-terminal Cys residue. In some embodiments, the carrier molecule is selected from a Drosophila Antennapedia homeodomain-derived sequence (CRQIKIWFQNRRMKWKK, SEQ ID NO: 84), a Transactivating Regulatory Protein (Tat)-derived transport polypeptide from the Human Immunodeficiency Virus, Type 1 (YGRKKRRQRRR, SEQ ID NO: 85), or a polyarginine, e.g., a sequence of arginine amino acid residues having a length sufficient to facilitate transport of the δPKC peptide into a cell. Suitable lengths are known in the art, and can be from, for example 5-15 residues in length, or 7-25, or 8-30.

In another aspect, a method is provided for reducing the incidence of hypertension-induced stroke or hypertension-induced encephalopathy, comprising administering to hypertensive mammalian patient an amount of δPKC inhibitor.

In another aspect, a kit of parts is provided for inhibiting disruption of the blood-brain barrier, comprising an amount of δPKC inhibitor and instructions for administering the δPKC inhibitor. In some embodiments, the kit of parts for reducing cerebral damage in hypertension-induced stroke or encephalopathy, comprises an amount of δPKC inhibitor and instructions for administering the δPKC inhibitor.

In addition to the exemplary aspects and embodiments described above, further aspects and embodiments will become apparent by reference to the sequences and by study of the following description.

### DETAILED DESCRIPTION

### I. Definitions

Unless otherwise indicated, all terms should be given their ordinary meaning as known in the art. See, e.g., Ausubel, F. M. et al., John Wiley and Sons, Inc., Media Pa., for definitions and terms of art. Abbreviations for amino acid residues are the standard 3-letter and/or 1-letter codes used in the art to refer to one of the 20 common L-amino acids.

As used herein and in the appended claims, the singular forms “a”, “an”, and “the” include plural reference unless the context clearly dictates otherwise.

As used herein, the “blood-brain barrier” refers to a naturally-occurring, anatomical-physiological barrier created by the modification of brain capillaries (e.g., by reduction in fenestration and formation of tight cell-to-cell contacts) to create a network of tightly-sealed blood vessels in the brain. The blood-brain barrier (abbreviated “BBB”) separates the parenchyma of the central nervous system from blood, thereby preventing or slowing the passage of various chemical compounds, radioactive ions, and disease-causing organisms, from the blood into the central nervous system.

As used herein, “reducing cerebral damage” means reducing cell death in a tissue of the central nervous system (CNS), particularly the brain. Reducing cell death includes reducing necrosis and/or reducing apoptosis. The cells of the CNS may be neuronal cells or supporting cells, such as astrocytes. Reducing cerebral damage includes reducing cell death in a tissue of the CNS by at least 25%, at least 50%, at least 75%, at least 85%, at least 95%, and even 100%, relative to a tissue that is untreated. Reducing cerebral damage includes preventing cerebral damage, preventing the worsening of cerebral damage, and/or reducing cerebral damage, compared to that in a control animal. In this manner, reducing cerebral damage refers to an absolute reduction or a relative reduction in the amount of cerebral damage.

As used herein “stroke” is a sudden focal neurological deficit caused by vascular insult, accompanied by cell damage in the central nervous system. Stroke may be caused by cerebral embolism, hemorrhage, cerebral thrombosis, or the like. A cerebral embolism involves blockage by an embolus (usually a clot) swept into an artery in the brain. Rupture of a blood vessel in or near the brain may cause an intracerebral hemorrhage or a subarachnoid hemorrhage. Cerebral thrombosis results from blockage by a thrombus (clot) that has built up on the wall of a brain artery.

Since any part of the brain may be affected by a stroke, the symptoms of stroke vary accordingly. Generally, symptoms of a stroke develop over minutes or hours, but occasionally over several days. Depending on the site, cause, and extent of damage, any or all of the symptoms of headache, dizziness and confusion, visual disturbance, slurred speech or loss of speech, and/or difficult swallowing, may be present. In more serious cases, a rapid loss of consciousness, coma, and death can occur, or severe physical or mental handicap may result. Certain factors increase the risk of stroke. One of the more important risk factors is hypertension, or high blood pressure.

As used herein, “hypertension” refers to sustained elevated blood pressure in the main arteries of the body. Blood pressure normally increases as a normal physiological response to stress and physical activity. However, a person with hypertension has a high blood pressure at rest. Hypertension or high blood pressure refers to a resting blood pressure (e.g., as measured with a sphygmomanometer), of greater than about 120 mmHg (systolic)/80 mmHg (diastolic). Blood pressure between 121-139/81-89 is considered prehypertension and above this level (140/90 mm Hg or higher) is considered high, or severe, (hypertension), which may also be defined in Stages (see the Table, below). Both prehypertension and severe hypertension are included in the meaning of “hypertension,” unless specified otherwise herein. For example, resting blood pressures of 135 mmHg/87 mm Hg or of 140 mmHg/90 mmHg are intended to be within the scope of the term “hypertension” even though the 135/87 is within a prehypertensive category. Blood pressures of 145 mm Hg/90 mmHg, 140 mmHg/95 mmHg, and 142 mmHg/93 mmHg are further examples of high blood pressures. It will be appreciated that blood pressure normally varies throughout the day. It can even vary slightly with each heartbeat. Normally, it increases during activity and decreases at rest. It's often higher in cold weather and can rise when under stress. More accurate blood pressure readings can be obtained by daily monitoring blood pressure, where the blood pressure reading is taken at the same time each day to minimize the effect that external factors. Several readings over time may be needed to determine whether blood pressure is high.

Persons suffering from chronic arterial hypertension typically have an unimpaired oxygen consumption and cerebral blood flow in the resting state. Hypertension may be primary (in which secondary causes such as renovascular disease, renal failure, pheochromocytoma, aldosteronism, or mendelian forms are not present) or secondary, meaning that the hypertension may be caused by another factor, such as those noted parenthetically. Increased blood pressure is associated with a number of risk factors, including obesity, insulin resistance, high levels of alcohol consumption, aging, sedentary lifestyle, stress, high levels of salt intake, low levels of potassium intake, and low levels of calcium intake.

As used herein, “hypertension-induced stroke” means stroke caused (in whole or in part) or increased in severity by hypertension.

As used herein, “hypertension-induced encephalopathy” is a disorder characterized by the failure of autoregulation, the breakdown of the blood-brain barrier, and/or the extravasation of fluid and/or protein into the brain parenchyma. The condition is commonly caused by a sustained elevation of systemic blood pressure.

“Ischemia” is defined as an insufficient supply of blood to a specific organ or tissue. A consequence of decreased blood supply can be an inadequate supply of oxygen to the organ or tissue (hypoxia). Prolonged hypoxia may result in injury to the affected organ or tissue.

“Anoxia” refers to a virtually complete absence of oxygen in the organ or tissue, which, if prolonged, may result in death of the organ or tissue.

“Hypoxic condition” is defined as a condition under which a particular organ or tissue receives an inadequate supply of oxygen.

“Anoxic condition” refers to a condition under which the supply of oxygen to a particular organ or tissue is cut off.

“Ischemic injury” refers to cellular and/or molecular damage to an organ or tissue as a result of a period of ischemia.

As used herein a “conserved set” of amino acids refers to a contiguous sequence of amino acids that is identical or closely homologous (e.g., having only conservative amino acid substitutions) between members of a group of proteins. A conserved set may be anywhere from 5-50 amino acid residues in length, more preferably from 6-40, still more preferably from 8-20 or 8-15 residues in length.

As used herein, a “conservative amino acid substitutions” are substitutions that do not result in a significant change in the activity or tertiary structure of a selected polypeptide or protein. Such substitutions typically involve replacing a selected amino acid residue with a different residue having similar physico-chemical properties. For example, substitution of Glu for Asp is considered a conservative substitution since both are similarly-sized negatively-charged amino acids. Groupings of amino acids by physico-chemical properties are known to those of skill in the art.

As used herein, the terms “domain” and “region” are used interchangeably herein and refer to a contiguous sequence of amino acids within a PKC isozyme, typically characterized by being either conserved or variable.

As used herein, the terms “peptide” and “polypeptide” are used interchangeably herein and refer to a compound made up of a chain of amino acid residues linked by peptide bonds. Unless otherwise indicated, the sequence for peptides is given in the order from the “N” (or amino) termiums to the “C” (or carboxyl) terminus.

Two amino acid sequences or two nucleotide sequences are considered “homologous” or are said to share a certain percent “identity” if they have an alignment score of >5 (in standard deviation units) using the program ALIGN with the mutation gap matrix and a gap penalty of 6 or greater (Dayhoff, M. O., in Atlas of Protein Sequence and Structure (1972) Vol. 5, National Biomedical Research Foundation, pp. 101-110, and Supplement 2 to this volume, pp. 1-10.) The two sequences (or parts thereof) are more preferably homologous if their amino acid sequences are greater than or equal to 50%, more preferably 70%, 75%, 80%, 85%, 90%, 95%, 96%, 97%, 98%, or 99% identical when optimally aligned using the ALIGN program mentioned above.

A peptide or peptide fragment is “derived from” a parent peptide or polypeptide if it has an amino acid sequence that is homologous to the amino acid sequence of, or is a conserved fragment from, the isolated parent peptide or polypeptide.

“Modulate” intends a lessening, an increase, or some other measurable change, e.g., in the permeability of the BBB or the incidence or severity of hypertension-induced stroke, hypertension-induced encephalopathy, or a related hypertension-related disorder.

“Management,” intends a lessening of the incidence or severity of the symptoms associated with hypertension-induced stroke, hypertension-induced encephalopathy, or a related hypertension-related disorder.

The term “treatment” or “treating” means any treatment of disease in a mammal, including: (a) preventing or protecting against the disease, that is, causing the clinical symptoms not to develop; (b) inhibiting the disease, that is, arresting or suppressing the development of clinical symptoms; and/or (c) relieving the disease, that is, causing the regression of clinical symptoms. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

The term “effective amount” means a dosage sufficient to provide treatment for the disorder or disease state being treated. This will vary depending on the patient, the disease and the treatment being effected.

The term “pharmaceutically acceptable carrier” or “pharmaceutically acceptable excipient” includes any and all solvents, dispersion media, vehicles, etc. suitable for administration to a mammal and generally known to a skilled artisan in the pharmaceutical arts. The use of such media and agents for pharmaceutically active substances is well known in the art. Supplementary active ingredients can also be incorporated into the compositions.

The following abbreviations are defined for clarity:

| Abbreviation | Meaning |
|---|---|
| l | liter |
| ml | milliliter |
| μl | microliter |
| M | molar |
| mM | millimolar |
| μM | micromolar |
| nM | nanomolar |
| pM | picomolar |
| g | gram |
| mg | milligram |
| μg | microgram |
| a.a. | amino acid |
| Min | minute(s) |
| sec or s | second(s) |
| Wks | weeks |
| α | alpha |
| β | beta |
| δ | delta |
| ε | epsilon |
| Sal | usually saline |
| N | number |
| h or hr | hour |

### II. Methods of Treatment

In a healthy subject, the BBB provides a tightly-sealed network of blood vessels that protect the brain from exposure to deleterious substances. Disruptions to the BBB wherein the integrity of the BBB is compromised (as evidenced for example by an increased permeability to a selected compound) can lead to undesired influx of inflammatory cells and mediators and pathogens, as well as edema and hemorrhage. The present methods provide a strategy for maintaining the integrity of the BBB in patients at risk of stroke, hypertension-induced stroke, or hypertension-induced encephalopathy. The treatment methods described herein also provide an approach for avoiding, preventing and/or inhibiting the disruption of the BBB in such patients. In these methods, a person diagnosed with hypertension, and therefore at risk of stroke or encephalopathy, is administered a compound having activity to inhibit cellular translocation of delta-PKC (δPKC). The administration can be a prophylactic treatment regimen or can be reactionary, e.g., administered during or subsequent to a stroke or encelphalopathic event.

In a study performed in support of the current method of treatment, hypertensive animals were treated with an inhibitor of δPKC and the effect of the compound on the integrity of the BBB was evaluated. The integrity of the BBB was evaluated by measuring its permeability to a dye and by a proteomic analysis of brain samples for levels of transferring and actin.

As detailed in Example 1, Dahl salt-sensitive (DS) rats, which are a model for hypertension in humans, were used to evaluate the efficacy of δPKC inhibitor treatment. The animals were initially maintained on a low-salt (0.3% NaCl) diet then switched to a high-salt (8% NaCl) to induce hypertension. The rats were subcutaneously treated with either a saline solution, a control peptide (TAT, SEQ ID NO:85), or a δPKC peptide inhibitor. Blood pressure was measured bi-weekly and blood-brain barrier (BBB) disruption was assessed at the end of the treatment period. Proteomic analysis of brain samples was carried out to identify differences in the brains of control TAT peptide and δV1-1-treated animals.

Although blood pressure was similar in the control TAT peptide and δPKC inhibitor-treated animals, the incidence of stroke (and stroke-associated symptoms) was significantly decreased in the δPKC inhibitor-treated animals relative to the TAT control group (n=24, p<0.01). Moreover, δPKC inhibitor treatment reduced Evans Blue extravasation into the brain parenchyma (n=6, p<0.01), indicating decreased BBB permeability. Proteomic analysis of brain samples showed increased levels of transferrin and actin in control animals, which were attenuated with continuous administration of the δPKC inhibitor. Furthermore, the administration of the δPKC inhibitor blocked the hypertension-induced redistribution of the tight junction-related proteins, zonula occludens 1 (ZO-1), and occludin, indicating that δPKC inhibitor-treatment stabilized the tight junctions. Together, these findings suggest that a δPKC inhibitor provides protection against hypertension-induced stroke and encephalopathy, at least in part by preventing BBB disruption.

Accordingly, in one embodiment, a method for maintaining the integrity of the BBB in hypertensive animals is contemplated, the method involving administering to the animal an inhibitor of δPKC. In one embodiment, the δPKC inhibitor is administered to a hypertensive patient at risk of stroke or encephalopathy. In another embodiment, the δPKC inhibitor is administered to a hypertensive patient at risk of a second or subsequent stroke following a first stroke episode.

Damage to the brain in DS rats is largely mediated by BBB disruption and edema. Therefore the administration of δPKC inhibitors appears to protect the brain from the pathologic changes that are associated with hypertension-induced stroke and encephalopathy. Accordingly, and in another embodiment, a method for reducing the extent of cellular damage due to a stroke episode or due to encephalopathy is provided, wherein a δPKC inhibitor is administered to the hypertensive subject before, during or after a stroke episode or encephalopathy.

### III. Exemplary δPKC Inhibitors

In the study described above, sustained delivery of the δPKC inhibitor compound provided significant protection against (BBB) disruption in hypertension-induced stroke and encephalopathy. δPKC inhibitors are safe and easy to administer, making them well-suited for the treatment of mammalian patients with hypertension, or who are at risk for developing hypertension, which is known to be a contributing factor to stroke and encephalopathy. A wide variety of inhibitors of δPKC may be utilized to reduce hypertension-induced stroke and encephalopathy. As used herein, inhibitors of δPKC are compounds that inhibit at least one biological activity or function of δPKC. Inhibitors suitable for use with the present invention may inhibit the enzymatic activity of δPKC, e.g., by preventing activation, preventing binding to and/or phosphorylation of a protein substrate, preventing binding to the receptor for activated kinase (RACK), and/or modulating the subcellular translocation of δPKC.

In certain embodiments, a protein inhibitor of δPKC is utilized. The protein inhibitor may be in the form of a peptide. Proteins, polypeptides, and peptides (used without distinction with respect to δPKC inhibitors) are known in the art, and generally refer to compounds comprising amino acid residues linked by peptide bonds. Unless otherwise stated, the individual sequence of the peptide is given in the order from the amino terminus to the carboxyl terminus. Polypeptide/peptide inhibitors of δPKC may be obtained by methods known to the skilled artisan. For example, the peptide inhibitor may be chemically synthesized using various solid phase synthetic technologies known to the art and as described, for example, in Williams, Paul Lloyd, et al. Chemical Approaches to the Synthesis of Peptides and Proteins, CRC Press, Boca Raton, Fla., (1997).

Alternatively, the peptide inhibitor may be produced by recombinant technology methods as known in the art and as described, for example, in Sambrook et al., Molecular Cloning: A Laboratory Manual, Cold Springs Harbor laboratory, 2^{nd} ed., Cold Springs Harbor, N.Y. (1989), Martin, Robin, Protein Synthesis: Methods and Protocols, Humana Press, Totowa, N.J. (1998) and Current Protocols in Molecular Biology (Ausubel et al., eds.), John Wiley & Sons, which is regularly and periodically updated. An expression vector may be used to produce the desired peptide inhibitor in an appropriate host cell and the product may then be isolated by known methods. The expression vector may include, for example, the nucleotide sequence encoding the desired peptide wherein the nucleotide sequence is operably linked to a promoter sequence.

As defined herein, a nucleotide sequence is “operably linked” to another nucleotide sequence when it is placed in a functional relationship with another nucleotide sequence. For example, if a coding sequence is operably linked to a promoter sequence, this generally means that the promoter may promote transcription of the coding sequence. Operably linked means that the DNA sequences being linked are typically contiguous and, where necessary to join two protein coding regions, contiguous and in reading frame. However, since enhancers may function when separated from the promoter by several kilobases and intronic sequences may be of variable length, some nucleotide sequences may be operably linked but not contiguous. Additionally, as defined herein, a nucleotide sequence is intended to refer to a natural or synthetic linear and sequential array of nucleotides and/or nucleosides, and derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a polypeptide.

The δPKC inhibitor may be derived from the delta (δ)-isozyme of PKC from any species, such as Rattus norvegicus (Genbank Accession No. AAH76505; SEQ ID NO: 86) or Homo sapiens (Genbank Accession No. NP_{—}997704; SEQ ID NO: 87). Exemplary δPKC inhibitors include δV1-1, having a portion of the amino acid sequence of δPKC from Rattus norvegicus (i.e., SFNSYELGSL; SEQ ID NO:1); δV1-2, having the sequence ALTTDRGKTLV (SEQ ID NO: 2), representing amino acids 35 to 45 of rat δPKC as found in Genbank Accession No. AAH76505; δV1-5, having the sequence KAEFWLDLQPQAKV (SEQ ID NO: 3), representing amino acids 101 to 114 of rat δPKC as found in Genbank Accession No. AAH76505; δV5, having the sequence PFRPKVKSPRPYSNFDQEFLNEKARLSYSDKNLIDSMDQSAF AGFSFVNPKFEHLLED (SEQ ID NO: 4), representing amino acids 569-626 of human δPKC as found in Genbank Accession No. BAA01381 (with the exception that amino acid 11 (aspartic acid) is substituted with a proline); and/or some combination of δV1-1, δV1-2, δV1-5 and δV5, including chimeras, variants, derivatives, or consensus sequences, thereof.

The δPKC peptide δV1-7, having the amino acid sequence MRAAEDPM (SEQ ID NO: 88), is an activator or δPKC. While unlikely to be of benefit in reducing hypertension-induced stroke and encephalopathy, δV1-7 (as well as variants, derivatives, or consensus sequences, thereof) is likely to be useful for inducing or increasing the severity of hypertension-induced stroke and encephalopathy, which may be of value in exacerbating hypertension in an experimental model.

The δPKC peptide inhibitors may include natural amino acids, such as the L-amino acids or non-natural amino acids, such as D-amino acids. The amino acids in the peptide may be linked by peptide bonds or, in modified peptides described herein, by non-peptide bonds.

A wide variety of modifications to the amide bonds which link amino acids may be made and are known in the art. Such modifications are discussed in general reviews, including in Freidinger, R. M. (2003) J. Med. Chem. 46:5553, and Ripka, A. S, and Rich, D. H. (1998) Curr. Opin. Chem. Biol. 2:441. These modifications are designed to improve the properties of the peptide by increasing the potency of the peptide or by increasing the half-life of the peptide.

The potency of the δPKC peptide inhibitor may be increased by restricting the conformational flexibility of the peptide. This may be achieved by, for example, including the placement of additional alkyl groups on the nitrogen or alpha-carbon of the amide bond, such as the peptoid strategy of Zuckerman et al, and the alpha modifications of, for example Goodman, M. et. al. ((1996) Pure Appl. Chem. 68:1303). The amide nitrogen and alpha carbon may be linked together to provide additional constraint (Scott et al. (2004) Org. Letts. 6:1629-1632).

The half-life of the δPKC peptide inhibitor may be increased by introducing non-degradable moieties to the peptide chain. This may be achieved by, for example, replacement of the amide bond by a urea residue (Patil et al. (2003) J. Org. Chem. 68:7274-80) or an aza-peptide link (Zega and Urleb (2002) Acta Chim. Slov. 49:649-62). Other examples of non-degradable moieties that may be introduced to the peptide chain include introduction of an additional carbon (“beta peptides”, Gellman, S. H. (1998) Acc. Chem. Res. 31:173) or ethene unit (Hagihara et al (1992) J. Am. Chem. Soc. 114:6568) to the chain, or the use of hydroxyethylene moieties (Patani, G. A. and Lavoie, E. J. (1996) Chem. Rev. 96:3147-76) and are also well known in the art. Additionally, one or more amino acids may be replaced by an isosteric moiety such as, for example, the pyrrolinones of Hirschmann et al. ((2000) J. Am. Chem. Soc. 122:11037), or tetrahydropyrans (Kulesza, A. et al. (2003) Org. Letts. 5:1163).

The inhibitors may also be pegylated, which is a common modification to reduce systemic clearance with minimal loss of biological activity. Polyethylene glycol polymers (PEG) may be linked to various functional groups of δPKC peptide inhibitor polypeptides/peptides using methods known in the art (see, e.g., Roberts et al. (2002), Advanced Drug Delivery Reviews 54:459-76 and Sakane et al. (1997) Pharm. Res. 14:1085-91). PEG may be linked to, e.g., amino groups, carboxyl groups, modified or natural N-termini, amine groups, and thiol groups. In some embodiments, one or more surface amino acid residues are modified with PEG molecules. PEG molecules may be of various sizes (e.g., ranging from about 2 to 40 kDa). PEG molecules linked to δPKC peptide inhibitor may have a molecular weight about any of 2,000, 10,000, 15,000, 20,000, 25,000, 30,000, 35,000, 40,000 Da. PEG molecule may be a single or branched chain. To link PEG to δPKC peptide inhibitor, a derivative of PEG having a functional group at one or both termini may be used. The functional group is chosen based on the type of available reactive group on the polypeptide. Methods of linking derivatives to polypeptides are known in the art.

Although the peptides are described primarily with reference to amino acid sequences from Rattus norvegicus, it is understood that the peptides are not limited to the specific amino acid sequences set forth herein. Skilled artisans will recognize that, through the process of mutation and/or evolution, polypeptides of different lengths and having different constituents, e.g., with amino acid insertions, substitutions, deletions, and the like, may arise that are related to, or sufficiently similar to, a sequence set forth herein by virtue of amino acid sequence homology and advantageous functionality as described herein.

The peptide inhibitors described herein also encompass amino acid sequences similar to the amino acid sequences set forth herein that have at least about 50% identity thereto and function to inhibit tumor growth and/or angiogenesis. Preferably, the amino acid sequences of the peptide inhibitors encompassed in the invention have at least about 60% identity, further at least about 70% identity, preferably at least about 75% or 80% identity, more preferably at least about 85% or 90% identity, and further preferably at least about 95% identity, to the amino acid sequences set forth herein. Percent identity may be determined, for example, by comparing sequence information using the advanced BLAST computer program, including version 2.2.9, available from the National Institutes of Health. The BLAST program is based on the alignment method of Karlin and Altschul ((1990) Proc. Natl. Acad. Sci. USA 87:2264-68) and as discussed in Altschul et al. ((1990) J. Mol. Biol. 215:403-10; Karlin and Altschul (1993) Proc. Natl. Acad. Sci. USA 90:5873-77; and Altschul et al. (1997) Nucleic Acids Res. 25:3389-3402).

Conservative amino acid substitutions may be made in the amino acid sequences described herein to obtain derivatives of the peptides that may advantageously be utilized in the present invention. Conservative amino acid substitutions, as known in the art and as referred to herein, involve substituting amino acids in a protein with amino acids having similar side chains in terms of, for example, structure, size and/or chemical properties. For example, the amino acids within each of the following groups may be interchanged with other amino acids in the same group: amino acids having aliphatic side chains, including glycine, alanine, valine, leucine and isoleucine; amino acids having non-aromatic, hydroxyl-containing side chains, such as serine and threonine; amino acids having acidic side chains, such as aspartic acid and glutamic acid; amino acids having amide side chains, including glutamine and asparagine; basic amino acids, including lysine, arginine and histidine; amino acids having aromatic ring side chains, including phenylalanine, tyrosine and tryptophan; and amino acids having sulfur-containing side chains, including cysteine and methionine. Additionally, amino acids having acidic side chains, such as aspartic acid and glutamic acid, are considered interchangeable herein with amino acids having amide side chains, such as asparagine and glutamine.

Modifications to δV1-1 that are expected to produce a δPKC inhibitor for reducing hypertension-induced stroke and encephalopathy, include peptides (or their derivatives) having the following changes to SEQ ID NO: 1 (shown in lower case and/or underlined): tFNSYELGSL (SEQ ID NO: 5), aFNSYELGSL (SEQ ID NO: 6), SFNSYELGtL (SEQ ID NO: 7), including any combination of these three substitutions, such as tFNSYELGtL (SEQ ID NO: 8). Other potential modifications include SyNSYELGSL (SEQ ID NO: 9), SFNSfELGSL (SEQ ID NO: 10), SNSYdLGSL (SEQ ID NO: 11), and SFNSYELpSL (SEQ ID NO: 12).

Additional modifications that are expected to produce a peptide that functions according to the described methods include changes of one or two L to I or V, such as SFNSYEiGSv (SEQ ID NO: 13), SFNSYEvGSi (SEQ ID NO: 14), SFNSYELGSv (SEQ ID NO: 15), SFNSYELGSi (SEQ ID NO: 16), SFNSYEiGSL (SEQ ID NO: 17), SFNSYEvGSL (SEQ ID NO: 18), aFNSYELGSL (SEQ ID NO: 19), any combination of the above-described modifications, and other conservative amino acid substitutions described herein.

Fragments and modification of fragments of δV1-1 are also contemplated, including but not limited to YELGSL (SEQ ID NO: 20), YdLGSL (SEQ ID NO: 21), fdLGSL (SEQ ID NO: 22), YdiGSL (SEQ ID NO: 23), iGSL (SEQ ID NO: 24), YdvGSL (SEQ ID NO:25), YdLpsL (SEQ ID NO: 26), YdLgiL (SEQ ID NO: 27), YdLGSi (SEQ ID NO: 28), YdLGSv (SEQ ID NO: 29), LGSL (SEQ ID NO: 30), iGSL (SEQ ID NO: 31), vGSL (SEQ ID NO: 32), LpSL (SEQ ID NO: 33), LGiL (SEQ ID NO: 34), LGSi (SEQ ID NO: 35), LGSv (SEQ ID NO: 36).

Accordingly, as used herein, the term “a δV1-1 peptide” encompasses a peptide identified by SEQ ID NO:1 and to a peptide having an amino acid sequence having a specified percent identity to the amino acid sequence of SEQ ID NO:1, including but not limited to the peptides set forth above, as well as fragments of any of these peptides that retain the ability to reduce hypertension-induced stroke and encephalopathy, as exemplified by (but not limited to) SEQ ID NOs: 20-36.

Modifications to δV1-2 that are expected to produce a δPKC inhibitor for reducing hypertension-induced stroke and encephalopathy, include the following changes to SEQ ID NO: 2 shown in lower case and/or underlined: ALsTDRGKTLV (SEQ ID NO: 37), ALTsDRGKTLV (SEQ ID NO: 38), ALTTDRGKsLV (SEQ ID NO: 39), and any combination of these three substitutions, ALTTDRpKTLV (SEQ ID NO: 40), ALTTDRGrTLV (SEQ ID NO: 41), ALTTDkGKTLV (SEQ ID NO: 42), ALTTDkGkTLV (SEQ ID NO: 43), changes of one or two L to I, or V and changes of V to I, or L and any combination of the above. In particular, L and V can be substituted with V, L, I, R and D, E can be substituted with N or Q. One skilled in the art would be aware of other conservative substitutions that may be made to achieve other derivatives of δV1-2 in light of the description herein.

Accordingly, the term “a δV1-2 peptide” as further used herein refers to a peptide identified by SEQ ID NO:2 and to a peptide having an amino acid sequence having a specified percent identity described herein to the amino acid sequence of SEQ ID NO:2, including but not limited to the peptides set forth in SEQ ID NOS:37-43, as well as fragments of any of these peptides that retain the ability to reduce hypertension-induced stroke and encephalopathy.

Modifications to δV1-5 that are expected to produce a δPKC inhibitor for reducing hypertension-induced stroke and encephalopathy, include the following changes to SEQ ID NO:3 shown in lower case and/or underlined:

| | |
|---|---|
| rAEFWLDLQPQAKV, | (SEQ ID NO: 44) |
| KAdFWLDLQPQAKV, | (SEQ ID NO: 45) |
| KAEFWLeLQPQAKV, | (SEQ ID NO: 46) |
| KAEFWLDLQPQArV, | (SEQ ID NO: 47) |
| KAEyWLDLQPQAKV, | (SEQ ID NO: 48) |
| KAEFWiDLQPQAKV, | (SEQ ID NO: 49) |
| KAEFWvDLQPQAKV, | (SEQ ID NO: 50) |
| KAEFWLDiQPQAKV, | (SEQ ID NO: 51) |
| KAEFWLDvQPQAKV, | (SEQ ID NO: 52) |
| KAEFWLDLnPQAKV, | (SEQ ID NO: 53) |
| KAEFWLDLQPnAKV, | (SEQ ID NO: 54) |
| KAEFWLDLQPQAKi, | (SEQ ID NO: 55) |
| KAEFWLDLQPQAKl, | (SEQ ID NO: 56) |
| KAEFWaDLQPQAKV, | (SEQ ID NO: 57) |
| KAEFWLDaQPQAKV, | (SEQ ID NO: 58) |
| and | |
| KAEFWLDLQPQAKa. | (SEQ ID NO: 59) |

Fragments of δV1-5 are also contemplated, including: KAEFWLD (SEQ ID NO: 60), DLQPQAKV (SEQ ID NO: 61), EFWLDLQP (SEQ ID NO: 62), LDLQPQA (SEQ ID NO:63), LQPQAKV (SEQ ID NO: 64), AEFWLDL (SEQ ID NO: 65), and WLDLQPQ (SEQ ID NO: 66).

Modifications to fragments of δV1-5 are also contemplated and include the modifications shown for the full-length fragments as well as other conservative amino acid substitutions described herein. The term “a δV1-5 peptide” as further used herein refers to SEQ ID NO: 3 and to a peptide having an amino acid sequence having a specified percent identity described herein to an amino acid sequence of SEQ ID NO: 3, as well as fragments thereof that retain the ability to reduce hypertension-induced stroke and encephalopathy.

Modifications to δV5 that are expected to produce a δPKC inhibitor for reducing hypertension-induced stroke and encephalopathy, include making one or more conservative amino acid substitutions, including substituting: R at position 3 with Q; S at position 8 with T; F at position 15 with W; V at position 6 with L and D at position 30 with E; K at position 31 with R; and E at position 53 with D, and various combinations of these modifications and other modifications that can be made by the skilled artisan in light of the description herein.

Fragments of δV5 are also contemplated, and include, for example, the following: SPRPYSNF (SEQ ID NO: 67), RPYSNFDQ (SEQ ID NO: 68), SNFDQEFL (SEQ ID NO: 69), DQEFLNEK (SEQ ID NO: 70), FLNEKARL (SEQ ID NO: 71), LIDSMDQS (SEQ ID NO: 72), SMDQSAFA (SEQ ID NO: 73), DQSAFAGF (SEQ ID NO: 74), FVNPKFEH (SEQ ID NO: 75), KFEHLLED (SEQ ID NO: 76), NEKARLSY (SEQ ID NO: 77), RLSYSDKN (SEQ ID NO: 78), SYSDKNLI (SEQ ID NO: 79), DKNLIDSM (SEQ ID NO: 80), PFRPKVKS (SEQ ID NO: 81), RPKVKSPR (SEQ ID NO: 82), and VKSPRPYS (SEQ ID NO: 83).

Modifications to fragments of δV5 are also contemplated and include the modifications shown for the full-length fragments as well as other conservative amino acid substitutions described herein. The term “a δV5 peptide” as further used herein refers to SEQ ID NO: 4 and to a peptide having an amino acid sequence having the specified percent identity described herein to an amino acid sequence of SEQ ID NO: 4, as well as fragments thereof that retain the ability to reduce hypertension-induced stroke and encephalopathy.

Peptide inhibitors for use according to the described methods may include a carrier protein, such as a cell permeable carrier peptide, or other peptides that increase cellular uptake of the peptide inhibitor, for example, a Drosophila Antennapedia homeodomain-derived sequence which is set forth in SEQ ID NO: 84 (CRQIKIWFQNRRMKWKK), and may be attached to the inhibitor by cross-linking via an N-terminal Cys-Cys bond as discussed in Theodore, L. et al. (1995) J. Neurosci. 15:7158-67 and Johnson, J. A. et al. (1996) Circ. Res 79:1086. Alternatively, the inhibitor may be modified by a Transactivating Regulatory Protein (Tat)-derived transport polypeptide (such as from amino acids 47-57 of Tat shown in SEQ ID NO: 85; YGRKKRRQRRR) from the Human Immunodeficiency Virus, Type 1, as described in Vives et al. (1997) J. Biol. Chem., 272:16010-17; U.S. Pat. No. 5,804,604; and Genbank Accession No. AAT48070; or with polyarginine as described in Mitchell et al. (2000) J. Peptide Res. 56:318-25 and Rothbard et al. (2000) Nature Med. 6:1253-57. Examples of Tat-conjugate peptides are provided in Example 1. The inhibitors may be modified by other methods known to the skilled artisan in order to increase the cellular uptake of the inhibitors.

While the present treatment method has largely been described in terms of polypeptides/peptide inhibitors, the method includes administering to an animal in need of such treatment a polynucleotide encoding any of the polypeptide/peptide inhibitors described herein. Polynucleotide encoding peptide inhibitors include gene therapy vectors based on, e.g., adenovirus, adeno-associated virus, retroviruses (including lentiviruses), pox virus, herpesvirus, single-stranded RNA viruses (e.g., alphavirus, flavivirus, and poliovirus), etc. Polynucleotide encoding polypeptides/peptide inhibitors further include naked DNA or plasmids operably linked to a suitable promoter sequence and suitable of directing the expression of any of the polypeptides/peptides described, herein.

A variety of other compounds can act as inhibitors of δPKC and may be utilized according to the methods described herein In one embodiment, organic molecule inhibitors, including alkaloids, may be utilized. For example, benzophenanthridine alkaloids may be used, including chelerythrine, sanguirubine, chelirubine, sanguilutine, and chililutine. Such alkaloids can be purchased commercially and/or isolated from plants as known in the art and as described, for example, in U.S. Pat. No. 5,133,981.

The bisindolylmaleimide class of compounds may also be used as inhibitors of δPKC. Exemplary bisindolylmaleimides include bisindolylmaleimide I, bisindolylmaleimide II, bisindolylmaleimide III, bisindolylmaleimide IV, bisindolylmaleimide V, bisindolylmaleimide VI, bisindolylmaleimide VII, bisindolylmaleimide VIII, bisindolylmaleimide IX, bisindolylmaleimide X and other bisindolylmaleimides that are effective in inhibiting δPKC. Such compounds may be purchased commercially and/or synthesized by methods known to the skilled artisan and as described, for example, in U.S. Pat. No. 5,559,228 and Brenner et al. (1988) Tetrahedron 44:2887-2892. Anti-helminthic dyes obtained from the kamala tree and effective in inhibiting δPKC may also be utilized, including rottlerin, and may be purchased commercially or synthesized by the skilled artisan.

Preferred δPKC inhibitors demonstrate similar biological activities as those inhibitors described, e.g., δV1-1, for example, using the DS mouse model as above and in Example 1. δPKC inhibitors can be efficiently identified using in vitro assays, such as the immunoblot analysis and quantitation of soluble and particulate δPKC assay, peptide activation of PKC assayed by substrate phosphorylation, and inhibition of δPKC translocation.

### IV. Administration and Dosing of PKC Inhibitors

An osmotic pump was used to deliver the δPKC inhibitors to experimental animals (see above and the Example). The osmotic pump allowed a continuous and consistent dosage of δPKC inhibitors to be delivered to animals with minimal handling. While an osmotic pump can be used for delivering δPKC inhibitors to human or other mammalian patients, other methods of delivery are contemplated.

The δPKC inhibitors are preferably administered in various conventional forms. For example, the inhibitors may be administered in tablet form for sublingual administration, in a solution or emulsion. The inhibitors may also be mixed with a pharmaceutically-acceptable carrier or vehicle. In this manner, the δPKC inhibitors are used in the manufacture of a medicament for reducing hypertension-induced stroke and encephalopathy.

The vehicle may be a liquid, suitable, for example, for parenteral administration, including water, saline or other aqueous solution, or may be an oil or an aerosol. The vehicle may be selected for intravenous or intraarterial administration, and may include a sterile aqueous or non-aqueous solution that may include preservatives, bacteriostats, buffers and antioxidants known to the art. In the aerosol form, the inhibitor may be used as a powder, with properties including particle size, morphology and surface energy known to the art for optimal dispersability. In tablet form, a solid vehicle may include, for example, lactose, starch, carboxymethyl cellulose, dextrin, calcium phosphate, calcium carbonate, synthetic or natural calcium allocate, magnesium oxide, dry aluminum hydroxide, magnesium stearate, sodium bicarbonate, dry yeast or a combination thereof. The tablet preferably includes one or more agents which aid in oral dissolution. The inhibitors may also be administered in forms in which other similar drugs known in the art are administered, including patches, a bolus, time release formulations, and the like.

The inhibitors described herein may be administered for prolonged periods of time without causing desensitization of the patient to the inhibitor. That is, the inhibitors can be administered multiple times, or after a prolonged period of time including one, two or three or more days; one two, or three or more weeks or several months to a patient and will continue to cause an increase in the flow of blood in the respective blood vessel.

The inhibitors may be administered to a patient by a variety of routes. For example, the inhibitors may be administered parenterally, including intraperitoneally; intravenously; intraarterially; subcutaneously, or intramuscularly. The inhibitors may also be administered via a mucosal surface, including rectally, and intravaginally; intranasally; by inhalation, either orally or intranasally; orally, including sublingually; intraocularly and transdermally. Combinations of these routes of administration are also envisioned.

Suitable carriers, diluents and excipients are well known in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament). Some formulations may include carriers such as liposomes. Liposomal preparations include, but are not limited to, cytofectins, multilamellar vesicles and unilamellar vesicles. Excipients and formulations for parenteral and nonparenteral drug delivery are set forth in Remington, The Science and Practice of Pharmacy (2000).

The skilled artisan will be able to determine the optimum dosage. Generally, the amount of inhibitor utilized may be, for example, about 0.0005 mg/kg body weight to about 50 mg/kg body weight, but is preferably about 0.05 mg/kg to about 0.5 mg/kg. The exemplary concentration of the inhibitors used herein are from 3 mM to 30 mM but concentrations from below about 0.01 mM to above about 100 mM (or to saturation) are expected to provide acceptable results.

The amount of inhibitor is preferably sufficient to reduce the incidence and/or severity of hypertension-induced stroke and encephalopathy by at least about 5%, at least about 10%, preferably at least about 25%, further at least about 50%, more preferably at least about 75% and further at least about 100%, compared to the clinical condition prior to treatment or compared to untreated animals. The reduction in the incidence and/or severity of hypertension-induced stroke and encephalopathy corresponds to a decrease in BBB permeability to a selected compound, such as a dye, relative to the BBB permeability to the same compound in a normotensive subject.

While BBB permeability is not readily measured in a living animal, a variety of clinically relevant end-point measurement are available for assessing the efficacy of δPKC inhibitors, including survival, reduced blood pressure, exercise tolerance, haemodynamics, echocardiographic parameters, and quality of life measures. The mammalian patient to be treated is typically one in need of such treatment as determined by blood pressure measurements. The following Table provides guidelines for selecting a mammalian patient (particularly a human patient) in need of treatment with δPKC inhibitors, based on blood pressure.

| Condition | Blood pressure (mm Hg, systolic/diastolic) |
|---|---|
| Normal blood pressure | <120/<80 |
| Prehypertension | 120-139/80-89 |
| Hypertension | >140/>90 |
| Stage 1 Hypertension | 140-159/90-99 |
| Stage 2 Hypertension | >160/>100 |

Blood pressure is readily measured by established methods in a non-invasive manner. Blood pressure measurements are also useful for identifying a patient in need of treatment and for monitoring a patient's response to treatment with δPKC inhibitors. For example, in one embodiment, an amount of δPKC inhibitor is administered to lower a patient's blood pressure, ideally to the range for normal blood pressure, and optionally to a pressure level lower than the level prior to treatment. Home blood pressure monitors allow a patient to measure blood pressure frequently. The efficacy of treatment may also be monitored by the lessening of behavioral and physiological conditions associated with hypertension, which include vision changes, cyanosis, dizziness, confusion, tiredness, edema, angina-like chest pain, ear noise or buzzing, nausea and vomiting, and respiratory distress, including signs such as flaring nostrils and grunting.

In other embodiments, a therapeutically effective amount of the inhibitor may be also be the amount sufficient or necessary for reducing the permeability of the BBB, as measured directly, for example, by Evans Blue dye extravsation in animals (normotensive and hypertensive) administered known amounts of a δPKC inhibitor. The optimum therapeutic dose is then determined (e.g., extrapolated) for humans, based on the animal results.

The patient is typically a vertebrate and preferably a mammal, including but not limited to a human. Other animals which may be treated include farm animals (such as horse, sheep, cattle, and pigs); pets (such as cats, dogs); rodents, mice, rats, gerbils, hamsters, and guinea pigs; members of the order Lagomorpha (including rabbits and hares); and any other mammal that may benefit from such treatment.

δPKC inhibitors of the invention may be combined with conventional treatments for hypertension, including but not limited to angiotensin-converting enzyme (ACE) inhibitors (e.g., CAPOTEN (captopril), VASOTEC (enalapril), PRINIVIL and ZESTRIL (lisinopril), LOTENSIN (benazepril), MONOPRIL (fosinopril), ALTACE (ramipril), ACCUPRIL (quinapril), ACEON (perindopril), MAVIK (trandolapril), and UNIVASC (moexipril)); angiotensin II receptor blockers (ARBs) (e.g., COZAAR (losartan), DIOVAN (valsartan), AVAPRO (irbesartan), and ATACAND (candesartan)); diuretics (e.g., ESIDRIX (hydrochlorothiazide or HCTZ), LASIX (furosemide), BUMEX (bumetanide), DEMADEX (torsemide), ZAROXOLYN (metolazone), and ALDACTONE (spironolactone); beta-blockers (e.g., Sectral (acebutolol), TENORMIN (atenolol), KERLONE (betaxolol), ZEBETA (bisoprolol), COREG (carvedilol), NORMODYNE and TRANDATE (labetalol), LOPRESSOR and TOPROL-XL (metoprolol), CORGARD (nadolol), LEVATOL (penbutolol), VISKEN (pindolol), INDERAL and INDERAL LA (propanolol), BETAPACE (sotalol) and BLOCADREN (timolol)); and calcium channel blockers (e.g., NORVASC (amlodipine), PLENDIL (felodipine), DYNACIRC (isradipine), CARDENE (nicardipine), PROCARDIA XL and ADALAT (nifedipine), CARDIZEM, DILACOR, TIAZAC, and DILTIA XL (diltiazem), ISOPTIN, CALAN, VERELAN, and COVERA-HS (verapamil)).

### V. Compositions and Kits Comprising δPKC Inhibitors

The methods may be practiced using polypeptide/peptide and/or peptimimetic inhibitors of δPKC, some of which are identified herein. These compositions may be provided as a formulation in combination with a suitable pharmaceutical carrier, which encompasses liquid formulations, tablets, capsules, films, etc. The δPKC inhibitors may also be supplied in lyophilized form. The compositions are suitable sterilized and sealed for protection.

Such compositions may be a component of a kit of parts (i.e., kit). In addition to a PKC inhibitor composition, such kits may include administration and dosing instructions, instructions for identifying patients in need of treatment, and instructions for monitoring a patients' response to PKC inhibitor therapy. Where the PKC inhibitor is administered via a pump (as in the animal studies described, herein), the kit may comprise a pump suitable for delivering PKC inhibitors. The kit may also contain a syringe to administer a formulation comprising a δPKC inhibitor by a peripheral route.

Kits of parts may further comprise an apparatus for measuring blood pressure, such as a sphygmomanometer or other blood pressure measuring apparatus. Home blood pressure measuring apparatus are well-known in the art.

### EXAMPLE

The following example is provided to illustrate the methods described herein. Additional embodiments of the methods will apparent to one skilled in the art.

### Example 1

### In Vivo Protection of Blood Brain Barrier

The PKC peptides and TAT₄₇₋₅₇ were synthesized and conjugated via a Cys S—S bond as described previously (Chen, et al. (2001) Proc. Natl. Acad. Sci. USA 25:11114-19 and Inagaki, et al. (2003) Circulation 11:2304-07). Reference herein to treatment with a “δV1-1 peptide” or a “δV1-1 inhibitor peptide” intends the peptide inhibitor linked to a carrier peptide to facilitate cell permeability, such as Tat.

Male Dahl salt-sensitive (DS) rats were maintained on a low-salt (0.3% NaCl) diet until 6 weeks of age. The animals were then switched to a high-salt (8% NaCl) diet until 15 weeks of age. The animals were monitored daily for symptoms of systemic hypertension.

The rats were subcutaneously infused (i.e., treated) with a saline solution, a control TAT peptide (YGRKKRRQRRR, SEQ ID NO:85), or the peptide inhibitor δV1-1 (SFNSYELGSL, SEQ ID NO: 1) attached via an N-terminal disulfide bond to TAT peptide (YGRKKRRQRRR-CC-SFNSYELGSL, SEQ ID NO:89), at a rate of 5.0 μL/hour, using an osmotic minipump. Treatment began at 11 weeks of age and survival rate and neurological deficits were recorded through week 15. Blood pressure was measured bi-weekly and blood-brain barrier disruption was assessed at 13 weeks by measuring Evans Blue dye extravasation. Proteomic analysis of brain samples was carried out to identify differences in the brains of control TAT peptide and δV1-1-treated animals. Blood brain barrier-related proteins were evaluated by immunoblot analysis.

Approximately 60-70% of the rats placed on a high-salt diet exhibit stroke damage and hypertension encephalopathy. Cerebral function remained normal in DS rats that were maintained to the low-salt diet. Although blood pressure was identical in the TAT peptide and δV1-1 peptide-treated animals, the incidence of stroke (and stroke-associated symptoms) was significantly decreased in the δV1-1 peptide-treated animals relative to the TAT control group (n=24, p<0.01). Treatment with the δV1-1 peptide also reduced Evans Blue extravasation into the brain parenchyma (n=6, p<0.01), indicating decreased BBB permeability (see, e.g., Rosengren, L. et al. (1977) Acta Neuropathol. (Berl.) 38:149-52). Proteomic analysis of brain samples taken at 13 weeks of age showed increased levels of transferrin and actin (compared to normotensive rats), which was attenuated with continuous administration of δV1-1. Furthermore, δV1-1 blocked the hypertension-induced redistribution of the tight junction-related proteins, zonula occludens 1 (ZO-1), and occludin, indicating that δV1-1 treatment induced stabilization of tight junctions. These findings suggest that δV1-1 provides protection against hypertension-induced stroke and encephalopathy, by preventing BBB disruption associated with hypertension. That is, administration of an inhibitor of δPKC maintained the integrity of the blood-brain barrier in a hypertensive subject, as evidenced by a reduction in blood brain barrier permeability to a test agent, such as a dye. In a preferred embodiment, a hypertensive subject treated with an inhibitor of δPKC has a blood brain barrier permeability to a test dye that is within 5%, more preferably 10%, still more preferably 20%, of the blood brain barrier permeability of the test dye in a normotensive subject.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A method for inhibiting disruption of the blood-brain barrier, comprising administering to a patient suffering from hypertension an inhibitor of delta protein kinase C (δPKC).

2. The method of claim 1, wherein the inhibitor of δPKC is a peptide.

3. The method of claim 2, wherein the peptide is selected from the first variable region of δPKC.

4. The method of claim 2, wherein the peptide is a peptide having between about 5 and 15 contiguous residues from the first variable region of δPKC.

5. The method of claim 2, wherein the peptide has at least about 50% sequence identity with a conserved set of between about 5 and 15 contiguous residues from the first variable region of δPKC.

6. The method of claim 2, wherein the peptide has at least about 80% sequence identity with SFNSYELGSL (SEQ ID NO:1).

7. The method of claim 6, wherein the peptide is modified to include a carrier molecule.

8. The method of claim 6, wherein the peptide is modified to include an N-terminal Cys residue.

9. The method of claim 7, wherein the carrier molecule is selected from a Drosophila Antennapedia homeodomain-derived sequence (CRQIKIWFQNRRMKWKK, SEQ ID NO: 84), a Transactivating Regulatory Protein (Tat)-derived transport polypeptide from the Human Immunodeficiency Virus, Type 1 (YGRKKRRQRRR, SEQ ID NO: 85), or a polyarginine.

10. A method for reducing the incidence of hypertension-induced stroke or hypertension-induced encephalopathy in a patient suffering from hypertension, comprising administering to the patient an amount of δPKC inhibitor.

11. The method of claim 10, wherein the inhibitor of δPKC is a peptide.

12. The method of claim 11, wherein the peptide is selected from the first variable region of δPKC.

13. The method of claim 11, wherein the peptide is a peptide having between about 5 and 15 contiguous residues from the first variable region of δPKC.

14. The method of claim 11, wherein the peptide has at least about 50% sequence identity with a conserved set of between about 5 and 15 contiguous residues from the first variable region of δPKC.

15. The method of claim 11, wherein the peptide has at least about 80% sequence identity with SFNSYELGSL (SEQ ID NO:1).

16. The method of claim 15, wherein the peptide is modified to include a carrier molecule.

17. The method of claim 15, wherein the peptide is modified to include a terminal Cys residue.

18. The method of claim 15, wherein the peptide is modified to include an N-terminal Cys residue.

19. The method of claim 16, wherein the carrier molecule is selected from a Drosophila Antennapedia homeodomain-derived sequence (CRQIKIWFQNRRMKWKK, SEQ ID NO: 84), a Transactivating Regulatory Protein (Tat)-derived transport polypeptide from the Human Immunodeficiency Virus, Type 1 (YGRKKRRQRRR, SEQ ID NO: 85), or a polyarginine.

20. A kit of parts for maintaining integrity of the blood-brain barrier in a person suffering from hypertension, comprising an amount of δPKC inhibitor and instructions for administering the δPKC inhibitor.

21. A kit of parts for reducing cerebral damage in hypertension-induced stroke or encephalopathy, comprising an amount of δPKC inhibitor and instructions for administering the δPKC inhibitor.
